(19) United States
(12) Patent Application Publication
(10) Pub. No.: US 2016/0333652 A1
(43) Pub. Date: Nov. 17, 2016

(54) Reduction of Microorganisms in Drilling Fluid Using Controlled Mechanically Induced Cavitation

(71) Applicant: Hydro Dynamics, Inc., Rome, GA (US)

(72) Inventor: Douglas G. Mancosky, White, GA (US)

(21) Appl. No.: 15/154,124

(22) Filed: May 13, 2016

Related U.S. Application Data
(60) Provisional application No. 62/161,639, filed on May 14, 2015.

(51) Int. Cl. E21B 21/06; A61L 2/02; E21B 43/26

(52) U.S. Cl. CPC E21B 21/06; E21B 43/26; A61L 2/02; E21B 21/003

(57) ABSTRACT

A method is disclosed for eliminating or reducing greatly the occurrence of bacteria and other microorganisms in commercial drilling fluids without the use of biocides or other chemicals. The method includes heating the fluid to obtain an initial microbe kill and passing the heated drilling fluid through a controlled cavitation reactor to expose the fluid to highly energetic shock waves and pressure variations within a cavitation zone of the reactor. The reactor includes a cylindrical rotor rotatably mounted within a cylindrical housing. The rotor has bores formed through its peripheral surface and the cavitation zone is defined between the peripheral surface of the rotor and the inner surface of the housing. As the rotor is rotated at a rapid rate with drilling fluid being urged through the cavitation zone, continuous cavitation events are induced in the fluid within the bores and these cause shockwaves and pressure variations to propagate through drilling fluid within the cavitation zone. The flow rate and rotation rate are selected so that the shock waves and pressure variations are sufficiently energetic to destroy the cellular structure of bacteria and other microorganisms in the drilling fluid thereby exterminating them.

### REFERENCE TO RELATED APPLICATION

Priority is hereby claimed to the filing date of U.S. provisional patent application 62/161,639 filed on May 14, 2015 and entitled Reduction of Microorganisms in Drilling Fluid Using Controlled Mechanically Induced Cavitation.

### TECHNICAL FIELD

This invention relates generally to fossil fuel drilling and more specifically to problems associated with bacteria and other microorganisms that thrive in drilling fluids used in the fossil fuel drilling industry.

### BACKGROUND

Drilling fluids have long been used in the oil and gas drilling industry when tapping underground reservoirs of oil, brine, gas, and water. The term “drilling fluid” as used in this disclosure is intended to encompass all types of fluids used in drilling operations including, for example, fracking fluid used in modern fracking operations, friction reducers, and drilling muds to name a few. Thus “drilling fluids” should be construed to mean any and all types of fluids that may be pumped into a bore hole during drilling operations. Drilling fluids are conventionally circulated down a bore hole through a drill pipe and bit and are returned through the earthen bore hole to the surface. These fluids serve a variety of purposes including, for instance, lubrication and cooling of the drill pipe and bit, transportation of cuttings to the surface, sealing and holding in place the traversed walls of the bore hole, establishing a hydrostatic head of pressure preventing the escape of high pressure fluids from the traversed formations, helping to liberate oil and gas from shale formations in fracking, and performing numerous other functions.

Drilling fluids have improved in efficacy over the years and modern drilling fluids usually include a complex mixture of many chemicals and other substances such as weighing materials, primarily barite (barium sulfate) or hematite to increase density; dispersants including iron lignosulfonates to break up solid clusters; flocculants, primarily acrylic polymers, to cause suspended particles in the fluid to group together for easier removal; surfactants like fatty acids and soaps to defoam and emulsify the fluid; and fluid loss reducers such as organic polymers to limit the loss of drilling fluid into under-pressurized or high permeability rock and earth formations. In addition to these and other additives, drilling fluids usually include one or more biocides such as organic amines, chlorophenols, or formaldehydes to kill bacteria, microbes, and perhaps other biological microorganisms that find their way into and thrive in the drilling fluid. In this disclosure, the terms bacteria, microbes, and microorganisms may be used interchangeably to refer to any microscopic living organisms in drilling fluid.

It is important to control the bacterial content of the drilling fluids for a number of reasons including the fact that bacteria and microorganisms can sour the fluid making it unsuitable for re-use. Further, certain bacteria common in drilling operations that ultimately find their way into drilling fluids are so called sulphate-reducing bacteria (SRB). These bacteria produce the toxic gas hydrogen sulphide, which can cause numerous problems. For example, hydrogen sulphide is corrosive and often causes significant drillstring and casing damage.

It has been suggested that SRBs and other biological organisms are either introduced to the formation by drilling or water injection or that they might be indigenous and activated by the drilling process. In either event, it is important to minimize the number of or eliminate SRB's and other microorganisms in drilling fluid. Previously, this has been accomplished as mentioned above through the addition of biocides to the drilling fluid. While this can be somewhat successful, it poses an environmental problem since many biocides are considered toxic substances that must be removed and treated before being released into the environment. Plus, chemical biocides for controlling microorganisms are themselves expensive and adversely affect the cost of a drilling operation.

A need exists for an efficient and effective process for eliminating or at least controlling biological microorganisms in drilling fluids in a way that does not require the addition of biocides and chemicals to the fluids and produces no environmentally unfriendly byproducts during use. It is to the provision of such a process that the present invention is primarily directed.

### SUMMARY

Briefly described, a system and method for exterminating bacteria and other microorganisms in drilling fluid are disclosed. The system includes a reservoir such as a holding tank for the drilling fluid and a controlled cavitation reactor. A pump draws drilling fluid from the reservoir and delivers the fluid to the inlet of the controlled cavitation reactor. The reactor has a cylindrical housing defining an internal cylindrical rotor. The rotor is rotatably mounted within the housing and has cavitation bores extending through its peripheral surface. A space between the peripheral surface of the rotor and the inner surface of the housing defines a cavitation zone. Within the controlled cavitation reactor, the drilling fluid passes through the cavitation zone with the rotor spinning at a relatively high rate of rotation.

In the cavitation zone, the drilling fluid is subjected to highly energetic shock waves and intense pressure variations created by continuous cavitation events in the fluid within the bores of the rotor. The cavitation activity, and thus the intensity of the shock waves and pressure variations, is controlled by the flow rate of the fluid and the rotation rate of the rotor. The goal is that the shock waves and pressure variations be sufficiently energetic to tear apart the tissue of bacteria and other living microorganisms within the drilling fluid.

After the drilling fluid is treated, it moves out of the controlled cavitation reactor and is delivered back to the drilling fluid reservoir or pumped to an oil or gas well for immediate use. The drilling fluid may be cycled through the controlled cavitation reactor as many times as needed to achieve an arbitrarily low bacteria content in the fluid, thus preventing souring of the fluid, greatly reducing corrosion of drilling equipment, and allowing the drilling fluid to be reused. Side benefits may include enhanced emulsification of the fluid, agglomeration of suspended particles for removal, and generally more homogeneity of the drilling fluid slurry.

Accordingly, a system and method are now provided for reducing bacteria and other microorganisms in drilling fluid that addresses the problems and shortcomings of traditional chemical treatments. These and other features, objects, and advantages of the system and methodology of this disclosure will be better appreciated upon review of the detailed description set forth below taken in conjunction with the accompanying drawing figures, which are briefly described as follows.

### BRIEF DESCRIPTION OF THE DRAWINGS

FIG. 1 is a schematic diagram illustrating a system and method for reducing bacterial and microorganisms in drilling fluid that embodies principles of the present invention in one exemplary form.

FIG. 2 is a cross sectional view of the controlled cavitation reactor of the system illustrating inlet and outlet ports and the internal cylindrical rotor.

### DETAILED DESCRIPTION

Controlled cavitation reactors for treating certain fluid flows are known. U.S. Pat. Nos. 8,465,642; 8,430,968; 7,507,014; 7,360,755; and 6,627,784, all owned by the assignee of the present patent disclosure, discuss variations of these controlled cavitation reactors. These patents are hereby incorporated fully by reference. To the extent that the patents may describe in detail the configurations and functions of controlled cavitation reactors, these configurations and functions will not be described in great detail again here. Also incorporated by reference is the disclosure of a copending (at the time of filing) patent application owned by the assignee of the present application and entitled Abrasion Resistant Controlled Cavitation Reactor.

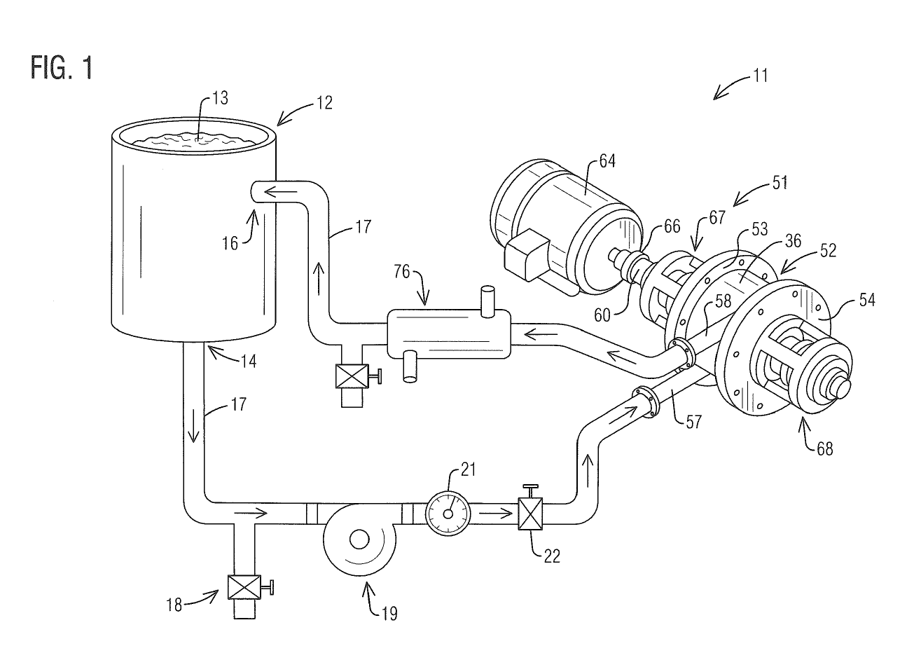

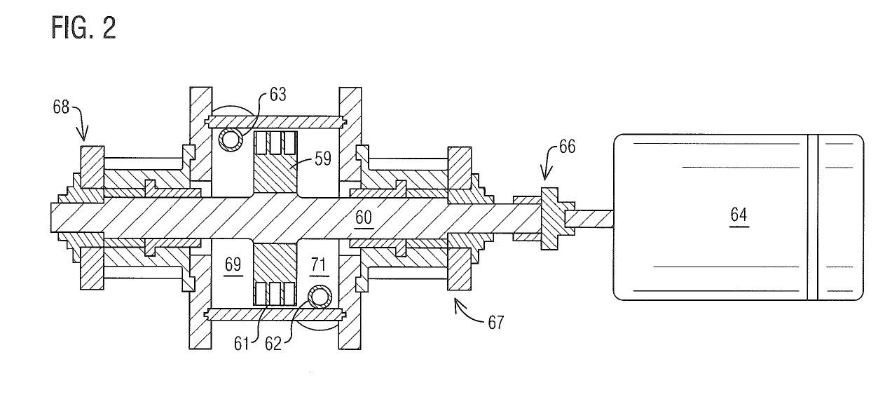

Referring now in more detail to FIGS. 1 and 2, an exemplary system is illustrated for treating drilling fluid to reduce or eliminate bacteria and other microorganisms within the fluid according to the methodology of the invention. The system 11, which is simplified for clarity in the figures, includes a reservoir such as a holding tank 12 for containing a volume of drilling fluid 13. An outlet 14 of the holding tank is coupled to a slurry pipe 17. A drain 18 may be incorporated in the slurry pipe 17 if desired for draining drilling fluid from the holding tank for delivery to a remote location or for use in a drilling operation.

A pump 19 is arranged in line with the slurry pipe 17 and is configured when activated to draw drilling fluid from the holding tank through the slurry pipe 17 and pump the fluid downstream of the pump. A flow meter 21 preferably is configured to monitor the flow rate of drilling fluid through the slurry pipe 17. In this way, the pump 19 can be controlled manually or with a computerized controller to establish a desired flow rate of drilling fluid through the system. A shut-off valve 22 may be incorporated in the slurry pipe 17 if desired.

The drilling fluid is delivered at a predetermined flow rate to a controlled cavitation reactor system 51. The controlled cavitation reactor system 51 includes a generally cylindrical housing 52 defined by a proximal end plate 53, a distal end plate 54, and a cylindrical peripheral wall 56. The end plates and the peripheral wall define a cylindrical interior cavity in the housing bounded by the inner surfaces of the end plates 53 and 54 and the inner surface of the peripheral wall 56. A cylindrical rotor 59 (FIG. 2) is mounted within the interior cavity of the housing 52 on a shaft 60, which, in turn, is journaled within bearing assemblies 67 and 68 on respective end plates of the housing. The shaft 60 is coupled through coupler 66 to an electric motor 64, which may be a variable frequency drive, such that the motor 64 can cause rotation of the rotor 59 within the housing at a desired and controllable rotation rate.

The peripheral surface of the rotor 59 is spaced from the inner surface of the peripheral wall 56 of the housing to define a cavitation zone 61 between the two. Cavitation bores are formed through the peripheral surface of the rotor. In the illustrated embodiment, the sides of the rotor and the end plates of the housing bound a proximal void zone 71 and a distal void zone 69. An inlet port 62 for receiving drilling fluid communicates with the housing within the proximal void zone 71. The inlet port preferably is arranged to introduce drilling fluid into the housing in a direction or along a path substantially tangential to the inner surface of the peripheral wall of the housing. An outlet port 63 communicates with the distal void zone 69, and is diametrically opposite to the inlet port 62 in this exemplary embodiment. The outlet port preferably is arranged to receive drilling fluid from the distal void zone in a direction or along a path substantially tangential to the inner surface of the peripheral wall of the housing.

In operation, the rotor 59 is rotated by the drive motor 64 in a counterclockwise direction when viewed from the distal end plate. Drilling fluid is introduced through the inlet port, urged under pressure through the cavitation zone, and is extracted through the outlet port. The tangential orientation of the inlet and outlet ports results in movement of drilling fluid into the reactor 52, through the cavitation zone 60, and out of the reactor 52 without making drastic changes in direction during the journey. This unique reactor configuration greatly reduces erosion of interior components of the controlled cavitation reactor caused by abrasive fluids. Abrasion resistance is desirable when treating drilling fluids since some drilling fluids can be abrasive in nature, containing small particles of sand, metal, and other materials. The method of this invention can be carried out by other controlled cavitation reactor designs without tangential inlets and outlets or with a different arrangement of tangential inlets and outlets, such as those disclosed in the incorporated patents. Accordingly, the scope of the methodology disclosed herein is not limited by the configuration of a particular controlled cavitation reactor or the placement and configuration of its inlet and outlet ports.

While in the cavitation zone 61, high energy cavitation events are continuously created in the fluid within the bores of the rotor. These cavitation events, in turn, generate highly energetic shock waves that propagate from the cavitation bores through the drilling fluid in the cavitation zone. The propagating shock waves result in extreme and very rapid pressure fluctuations within the drilling fluid. When the flow rate of the fluid, its temperature, and the rotation rate of the rotor are properly chosen, the dwell time of the fluid in the cavitation zone and the shock wave and pressure variation energy in the cavitation zone are sufficient to tear apart the cellular structure of bacteria and other microorganisms in the fluid, thereby destroying and exterminating them.

An advantage of the present invention it is that it should work at nearly any flow rate, rotor rotation rate, and temperature with corresponding varying degrees of efficiency. In other words, some microorganism mortality occurs at just about any combination of these factors. Typically a starting temperature of a drilling fluid is between about 32° F. and 70° F. Preferably the drilling fluid is heated to a higher temperature between about 75° F. and 150° F. before being treated in the controlled cavitation reactor. Maximum elimination of living microorganisms occurs at higher temperatures and/or higher delta temperatures, but some beneficial effect would be expected at lower fluid temperatures.

Effectiveness of the treatment of drilling fluid in the controlled cavitation reactor also can vary with the source and type of fluid and the physical robustness of living microorganisms found in differing locations and during different seasons. The best practice is to take the “free” microbial extermination that comes from pre-heating the drilling fluid to a target temperature, and then multiplying that microbial kill by choosing an appropriate flow rate and rotor RPM in the controlled cavitation reactor. Of course, it is not possible to kill every microbe that might reside within a drilling fluid, but the higher the microbe kill rate using the present invention, the lower the volume of antimicrobial chemicals needed even to the elimination of the need for such chemicals.

The molecular and particle agitation caused by the energetic pressure variations in the cavitation zone 61 result in friction in the drilling fluid. This, in turn, causes the temperature of the drilling fluid to rise further. As the treated and heated fluid exits the controlled cavitation reactor 52 through the outlet 58, it is directed in this embodiment to a heat exchanger 76. Here, excess heat imparted to the fluid in the controlled cavitation reactor can be extracted if required before the fluid is delivered through slurry pipe 17 back to the holding tank 12 or to a remote location for use. In the embodiment shown in FIG. 1, the drilling fluid from the reservoir can be cycled and recycled through the controlled cavitation reactor 52 as many times as necessary to obtain an arbitrarily low level of living bacteria and other microorganisms in the fluid. The fluid thus does not become soured and can be re-used without introducing corrosive bacterial byproducts into the well. These benefits are obtained without the use of or with greatly reduced use of anti-microbial chemicals and biocides which can be expensive and pose disposal and environmental problems when the drilling fluid is spent.

The invention has been described herein in terms of preferred embodiments and methodologies considered by the inventor to represent the best mode of carrying out the invention. It will be clear to the skilled artisan, however, that a wide gamut of additions, deletions, and modifications, both subtle and gross, may be made to the exemplary embodiments described above without departing from the spirit and scope of the invention. All such additions and deletions should be construed to fall within the scope of the invention.

What is claimed is:

1. A method of reducing or eliminating bacteria and other microorganisms in drilling fluid comprising the steps of establishing shock waves in the drilling fluid with the shock waves being generated by mechanically induced cavitation, the shock waves being of a sufficient energy, and the drilling fluid being subjected to the shock waves for a sufficient period of time, to destroy the cellular structure of and thereby exterminate bacteria and other microorganisms in the drilling fluid.

2. A method according to claim 1 wherein the step of establishing shock waves in the drilling fluid comprises passing the drilling fluid through a cavitation zone of a controlled cavitation reactor.

3. A method according to claim 2 wherein the step of passing the drilling fluid through a cavitation zone comprises establishing a cavitation zone between the interior of a housing and the peripheral surface of a rotor within the housing, urging the drilling fluid through the cavitation zone, and rotating the rotor to generate cavitation within the drilling fluid in the cavitation zone.

4. A method according to claim 3 wherein cavitation structures are formed in the peripheral surface of the rotor.

5. A method according to claim 4 wherein the cavitation structures comprise bores in the peripheral surface of the rotor and cavitation is induced in the drilling fluid within the bores.

6. A method according to claim 2 further comprising moving the drilling fluid through a void zone of the controlled cavitation reactor prior to passing the drilling fluid through the cavitation zone of the controlled cavitation reactor.

7. A method according to claim 6 wherein the void zone is defined between a side of the rotor and an interior wall of the housing.

8. A method according to claim 7 wherein the internal cavity of the housing is generally cylindrical and where the step of moving the drilling fluid through a void zone comprises moving the fluid into the void zone through an inlet port oriented substantially tangentially to the generally cylindrical cavity of the housing.

9. A method according to claim 6 further comprising moving the drilling fluid through a second void zone of the controlled cavitation reactor after passing the drilling fluid through the cavitation zone of the controlled cavitation reactor.

10. A method according to claim 9 wherein the second void zone is defined between a second side of the rotor and an interior wall of the housing.

11. A method according to claim 10 wherein the internal cavity of the housing is generally cylindrical and where the step of moving the drilling fluid through a second void zone comprises moving the fluid out of the second void zone through an outlet port oriented substantially tangentially to the generally cylindrical cavity of the housing.

12. A method according to claim 1 wherein the steps of the method are repeated a predetermined number of times to destroy the cellular structure of and thereby exterminate residual bacteria and other microorganisms in the drilling fluid.

13. A method of treating drilling fluid to eliminate live bacteria and other microorganisms in the drilling fluid, the method comprising the steps of:
(a) obtaining a controlled cavitation reactor having a housing defining a cylindrical interior cavity at least partially bounded by an inner surface of a peripheral wall, the controlled cavitation reactor further having a cylindrical rotor with opposed sides and a peripheral surface mounted within the interior cavity, the peripheral surface of the cylindrical rotor being provided with cavitation bores, a cavitation zone of the reactor being defined between the peripheral surface of the rotor and the inner surface of the peripheral wall;
(b) rotating the rotor within the housing at a predetermined rate of rotation;
(c) introducing drilling fluid into the housing through an inlet port of the controlled cavitation reactor;
(d) urging the drilling fluid to move through the cavitation zone of the controlled cavitation reactor;
(e) as the drilling fluid moves through the cavitation zone, inducing cavitation events in drilling fluid within the cavitation bores of the rotating rotor to generate shock waves and pressure variations that propagate through the drilling fluid in the cavitation zone;
(f) controlling the rate of rotation of the rotor and the rate at which the drilling fluid moves through the cavitation zone such that the shock waves and pressure variations generated in the fluid within the cavitation zone are of sufficient intensity to exterminate bacteria and other microorganisms in the drilling fluid within the cavitation zone; and
(g) extracting the drilling fluid from the housing through an outlet port of the controlled cavitation reactor.

14. The method of claim 13 wherein a void zone is defined between one of the opposed sides of the rotor and an interior surface of a wall of the housing, and wherein step (c) comprises introducing the drilling fluid into the housing within the void zone.

15. The method of claim 14 wherein step (c) further comprises introducing the drilling fluid into the housing along a path substantially tangent to the cylindrical peripheral wall of the housing.

16. The method of claim 13 wherein a second void zone is defined between the other one of the opposed sides of the rotor and an interior surface of a wall of the housing, and wherein step (g) comprises extracting the fluid from the housing within the second void zone.

17. The method of claim 16 wherein step (g) further comprises extracting the drilling fluid out of the housing along a path substantially tangent to the cylindrical peripheral wall of the housing.

18. The method of claim 13 further comprising repeating steps (c) through (g) a predetermined number of times to exterminate residual bacteria and other microorganisms in the drilling fluid.

19. The method of claim 13 further comprising heating the drilling fluid to a predetermined temperature sufficient to begin the extermination of bacteria and other microorganisms within the drilling fluid prior to step (c).
